(19) United States
(12) Patent Application Publication
(10) Pub. No.: US 2026/0174738 A1
(43) Pub. Date: Jun. 25, 2026

(54) COMPOSITIONS AND METHODS FOR INHIBITING TYPE I COLLAGEN PRODUCTION

(71) Applicant: Florida State University Research Foundation, Inc., Tallahassee, FL (US)

(72) Inventor: Branko Stefanovic, Tallahassee, FL (US)

(21) Appl. No.: 19/311,361

(22) Filed: Aug. 27, 2025

Related U.S. Application Data
(60) Provisional application No. 63/736,845, filed on Dec. 20, 2024.

(51) Int. Cl. A61K 31/426; C07D 277/587

(52) U.S. Cl. CPC A61K 31/426; C07D 277/587

(57) ABSTRACT

Nano-entity compositions composed of a plurality of self-associated compounds of Formula 1 and/or Formula 2 have antifibrotic properties. Such compositions may be used to inhibit type I collagen synthesis.

### CROSS REFERENCE TO RELATED APPLICATION

This claims the benefit of and priority to Application No. 63/736,845, filed Dec. 20, 2024, which is incorporated by reference in its entirety.

### FIELD

This is related to the field of type I collagen synthesis and, more particularly to, compounds inhibiting type I collagen synthesis.

### BACKGROUND

Collagen is a protein that gives bones, connective tissues, and organs their physical structure. Different forms of collagen are identified by their “types.” Type I collagen, the primary form found in the human body, is found in the skin, tendons, ligaments, bones, organs, and vascular system and has a fiber-like structure.

If the body produces excessive amounts of type I collagen, this can lead to a condition called fibrosis. Fibrosis is the presence of excess connective tissue in a region of the body. Excess connective tissue in an organ can interfere with the organ's functions. There are many diseases related to fibrosis, some of which include arterial hardening, cirrhosis, scleroderma, and myelofibrosis. Fibrosis-related diseases are a major medical problem throughout the world.

### BRIEF SUMMARY

New compositions that can be used to treat fibrotic conditions are needed. Such a composition comprises a pharmaceutical dosage form including a nano-entity composed of a plurality of self-associated compounds of the first formula

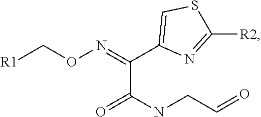

wherein R1 and R2 are independently selected from the group consisting of H, an alkyl group, an alkenyl group, an alkynyl group, an aryl group, an alkoxy group, a hydroxy group, a carboxy group, an acyl group, a nitro group, a phosphor group, a halo group, a sulfo group, an ester group, an ether group, an amine group, and a combination thereof.

This composition may include at least one of the following additional features.

R1 may be a carboxyl group.

R2 may be an amine group.

The pharmaceutical dosage form may also include a tautomer of the first formula.

The nano-entity may also be composed of a plurality of self-associated compounds of the second formula

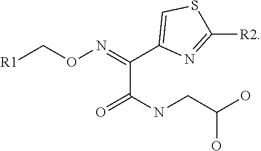

The first formula may be

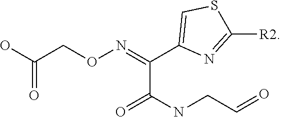

The nano-entity may also be composed of a plurality of self-associated compounds of the second formula

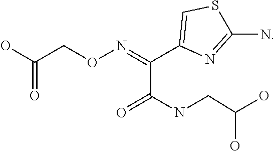

The pharmaceutical dosage form may be effective to inhibit collagen synthesis by inhibiting binding of LARP6 with a 5′ stem-loop of a collagen mRNA.

The pharmaceutical dosage form may be at least one of an oral dosage form and an injectable dosage form.

An example of a method comprises administering to a patient having a fibrotic condition a therapeutically effective amount of the composition describe above to the patient.

This method may include at least one of the following features.

The fibrotic condition may be at least one of a pulmonary fibrosis, a liver fibrosis, a heart fibrosis, a circulatory system fibrosis, a skin fibrosis, a renal fibrosis, and/or an intestinal fibrosis.

Administering may be by oral administration and/or by injection.

R1 may be a carboxyl group.

R2 may be an amine group.

The pharmaceutical dosage form may also include a tautomer of the first formula.

The nano-entity may also be composed of a plurality of self-associated compounds of the second formula

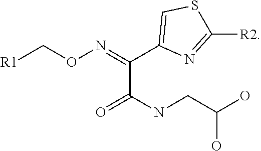

The first formula may be

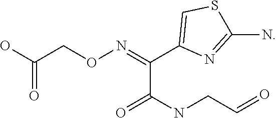

The nano-entity may also be composed of a plurality of self-associated compounds of the second formula

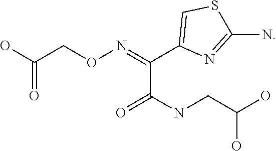

Another example of a method comprises performing an acid catalyzed hydrolysis of the first formula

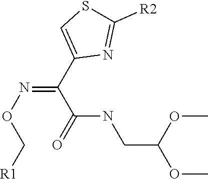

to form a tautomeric mixture of a second formula

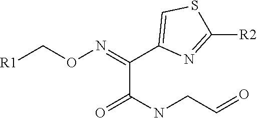

and a third formula

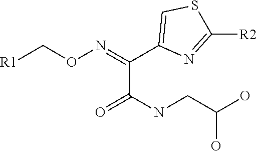

The method continues by dissolving the tautomeric mixture in a solvent to form a nano-entity composed of a plurality of self-associated compounds of the second formula and the third formula. R1 and R2 are independently selected from the group consisting of H, an alkyl group, an alkenyl group, an alkynyl group, an aryl group, an alkoxy group, a hydroxy group, a carboxy group, an acyl group, a nitro group, a phosphor group, a halo group, a sulfo group, an ester group, an ether group, an amine group, and a combination thereof.

This method may include at least one of the following additional features.

The method may further comprise precipitating the nano-entity and dissolving the precipitated nano-entity in an aqueous solution having a pH of 6-14.

The solvent may be dimethyl sulfoxide.

The acid catalyzed hydrolysis may be performed at a pH of 1-4 and a temperature of 45 C to 90 C.

### BRIEF DESCRIPTION OF THE DRAWINGS

FIG. 1 is a diagram of Formula 1.

FIG. 2 is a diagram of Formula 2.

FIG. 3 is a diagram of Formula 3.

FIG. 4 is a diagram of Formula 4.

FIG. 5 is a reaction diagram illustrating the acid catalyzed hydrolysis of Formula 5.

FIG. 6 is a diagram of Formula 6.

FIG. 7 is a graph of dynamic light scattering analysis of ATO-OA nano-entities (ATO-OA NE).

FIG. 8 is a table of the data of FIG. 7.

FIG. 9 is a graph of dynamic light scattering analysis with and without detergent.

FIG. 10 is a graph of dynamic light scattering analysis showing ATO-OA NE is stable at elevated temperatures.

FIG. 11 is data showing dose response inhibition of LAM (containing two domains of LARP6; LA and RRM) binding to collagen 5′SL RNA sequence present in COL1A1 mRNA by ATO-OA NE (left panel) and that ATO-OA NE activity is minimally affected by presence of detergent (right panel).

FIG. 12 is data showing a comparison of the binding inhibition of LAM to COL1A1 5′SL (left panel) and in COL1A2 mRNA (right panel).

FIG. 13 is data showing ATO-OA NE does not affect binding of LA domain alone to 5′SL RNA. The left panel is the dose response inhibition of LA binding to collagen 5′SL RNA sequence of COL1A1 mRNA by ATO-OA NE. The right panel is the dose response inhibition when LA and LAM are present simultaneously.

FIG. 14 is data showing that an excess of RRM domain can sequester ATO-OA NE and reverse the inhibition of LAM binding.

FIG. 15 is a graph showing that ATO-OA NE changes the conformation of the RRM domain (left panel), while its methyl acetal precursor (Formula 6) is inactive (right panel).

### DETAILED DESCRIPTION

Organ fibrosis is a major medical problem and primary and secondary fibrosis affect 45% of population. There are no approved true antifibrotic drugs and development of specific and potent antifibrotic probes with specific mechanism of action has economic potential in billions of dollars and will have millions of direct users.

The two drugs approved for treatment of idiopathic pulmonary fibrosis are: tyrosine kinase inhibitor nintedanib, and pirfenidone, the mechanism of which is not completely understood. There are no antifibrotic drugs directly targeting biosynthesis of type I procollagen. The experimental antifibrotic drugs currently under investigation are limited to inhibition of pleiotropic signaling pathways, what results in serious side effects upon prolonged application.

It has now been discovered that some compounds are effective for inhibiting type I collagen synthesis by cells and, therefore, may be used as antifibrotic drugs to treat fibrotic conditions. Such compounds are effective to decrease the binding affinity of LARP6 for the 5′ stem-loop of collagen mRNAs.

LARP6 regulates type I collagen expression in fibrosis, which is characterized by excessive synthesis of type I collagen in various organs. LARP6 binds a unique sequence present in type I collagen mRNAs: the 5′ stem-loop (5′SL). This binding regulates translation of type I collagen mRNAs and is necessary for fibrosis development in vivo. Mice in which binding of LARP6 to 5′SL is genetically abolished are resistant to liver fibrosis. This indicates that the inhibitors of LARP6 binding to collagen mRNAs can be specific and effective antifibrotic drugs.

U.S. Pat. No. 8,697,385 describes a method of screening a compound for its ability to interfere with collagen synthesis that takes advantage of this property of LARP6. Such a method may be used to screen different compounds for antifibrotic activity.

Described herein are compounds that can be used to inhibit the biosynthetic step which is specific for type I procollagen, namely, binding of LARP6 to 5′ stem-loop RNA sequence. In this manner, it directly inhibits the mechanism of profibrotic type I collagen biosynthesis.

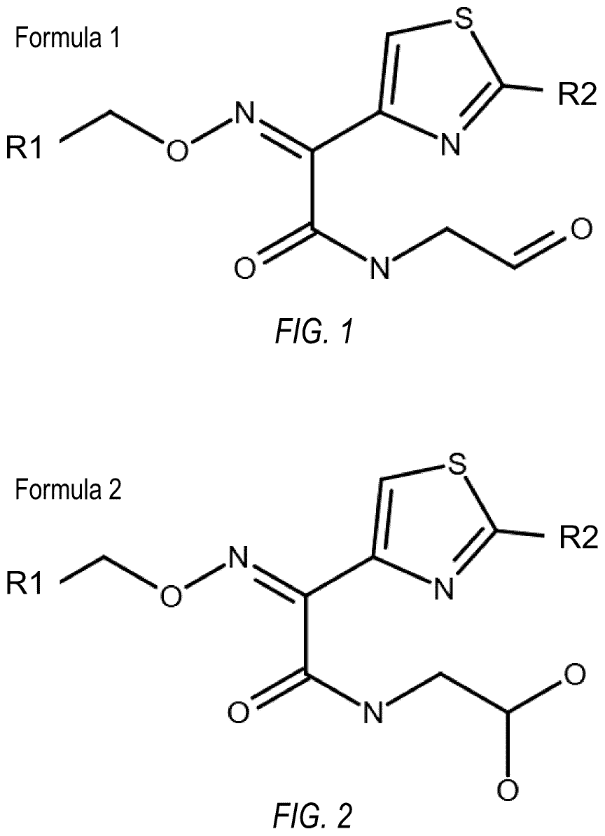

Referring to FIGS. 1 and 2, an example of two such compounds are shown as Formula 1 and Formula 2. The compound of Formula 2 is a hydride tautomer of Formula 1 that forms when Formula 1 is in an aqueous solution. Tautomers exist as two or more chemical compounds that are capable of interconversion. This often means the exchange of a hydrogen atom between two other atoms. Tautomers exist in equilibrium with each other, thus attempts to prepare the separate forms usually results in the formation of a tautomer mixture. In this example, the tautomers are Formula 1 and Formula 2.

The compounds of Formula 1 and Formula 2 have multiple hydrogen bond donors and acceptors, giving them the propensity to form hydrogen bonds between the molecules, which results in spontaneous formation of nano-entities.

Effective compounds of Formula 1 and 2 self-associate into multi-molecule units called nano-entities in which individual molecules interact via hydrogen bonding. These nano-entities resemble self-associated clusters of molecules of Formula 1 and Formula 2. Such nano-entities are effective to inhibit collagen synthesis by inhibiting binding of LARP6 with a 5′ stem-loop of a collagen mRNA.

Although the nano-entity size and number of molecules therein may vary, a particular example of the nano-entity has a hydrodynamic radius of 1.9 nm, which suggests the nano-entity contains 50 or fewer molecules of Formula 1 or Formula 2. The nano-entities do not seem to be colloidal aggregates.

R1 and R2 may independently be at least one member selected from the group H, an alkyl group, an alkenyl group, an alkynyl group, an aryl group, an alkoxy group, a hydroxy group, a carboxy group, an acyl group, a nitro group, a phosphor group, a halo group, a sulfo group, an ester group, an ether group, and an amino group.

The alkyl group may be a straight, cyclic, or branched chain alkane hydrocarbon residue containing 1 to 12 carbon atoms. The alkyl group may be a straight or branched chain hydrocarbon residue containing 1 to 7 carbon atoms. Examples of particular alkyl groups include methyl, ethyl, propyl, isopropyl, n-butyl, isobutyl, and tert-butyl. The alkyl group may be substituted with substituents.

The alkenyl group may be a straight, cyclic, or branched chain alkene hydrocarbon residue containing 1 to 12 carbon atoms. The alkenyl group may be a straight or branched chain alkene hydrocarbon residue containing 1 to 7 carbon atoms. Examples of particular alkenyl groups include methenyl, ethenyl, propenyl, isopropenyl, and butenyl. The alkenyl group may be substituted with substituents.

The alkynyl group may be a straight, cyclic, or branched chain alkyne hydrocarbon residue containing 2 to 12 carbon atoms. The alkynyl group may be a straight or branched chain alkyne hydrocarbon residue containing 2 to 7 carbon atoms. Examples of particular alkynyl groups include methynyl, ethynyl, propynyl, isopropyl, and butenyl. The alkenyl group may be substituted with substituents.

The aryl group may be a substituted phenyl or napthyl, for example. Aryl groups may include examples such as benzyl, tolyl, xylyl, and the like. Suitable substituents for aryl may be, for example, alkyl, halogen, hydroxy, and substituted alkyl, haloalkyl, alkenyl, alkynyl, and aryloxy groups.

The alkoxy group may be a substituted straight or branched chain alkyl-oxy group where the alkyl portion is defined above. Examples may include methoxy, ethoxy, n-propyloxy, i-propyloxy, n-butyloxy, i-butyloxy, tert-butyloxy, pentyloxy, hexyloxy, and heptyloxy.

In certain examples an acyl group may be a group containing R—C═O substituted with any of the other groups mentioned above.

Some compounds of Formula 1 and Formula 2 that are basic may form pharmaceutically acceptable salts with inorganic acids such as hydrohalic acids such as hydrochloric acid and hydrobromic acid, sulfuric acid, nitric acid and phosphoric acid, and the like, and with organic acids such as acetic acid, tartaric acid, succinic acid, fumaric acid, maleic acid, malic acid, salicylic acid, citric acid, methanesulfonic acid and p-toluene sulfonic acid, and the like. The formation and isolation of such salts can be conducted according to conventional methods for forming and isolating pharmaceutically acceptable salts.

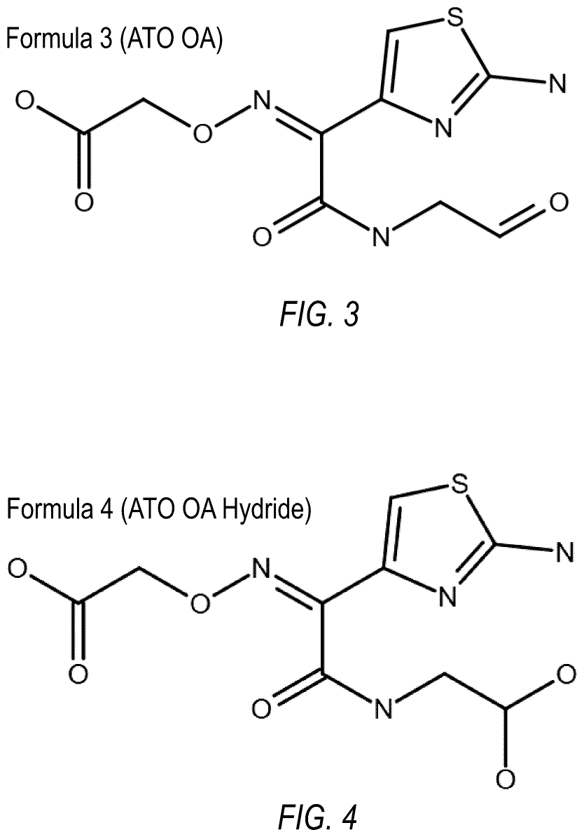

Referring to FIG. 3, a specific example of Formula 1, called Formula 3, is shown. R1 is the carboxylic acid group and R2 is the amine group. The compound in Formula 3 is 2-[(Z)-[1-(2-amino-1,3-thiazol-4-yl)-2-oxo-2-(2-oxoethylamino)ethylidene]amino]oxyacetic acid, abbreviated here as ATO-OA. The SMILES for Formula 3 is NC1=NC(═CS1)C(═N\OCC(O)═O)\C(═O)NCC═O.

Referring to FIG. 4, the hydride tautomer of Formula 3 is shown as Formula 4 with R1 being the carboxylic acid group and R2 being the amine group. The SMILES for Formula 4 is NC1=NC(═CS1)C(═N\OCC(O)═O)\C(═O)NCC(O)O. Formula 4 may be abbreviated herein as ATO OA Hydride.

The compounds of Formula 1 and 2, including those of Formula 3 and Formula 4, are effective to inhibit collagen synthesis by inhibiting binding of LARP6 with a 5′ stem-loop of a collagen mRNA is active when self-assembled into a nano-entity.

Any of the nano-entities of Formula 1-4 or any combination thereof may be administered as an active ingredient in a pharmaceutical dosage form composition.

The nano-entities may be combined with ingredients conventionally used in pharmaceutically acceptable dosage forms such as suspensions, tablets, capsules, injectables, dermal patches, or other dosage form.

Examples of ingredients used in such pharmaceutical dosage forms include excipients, diluents, disintegrants, emulsifiers, solvents, processing aids, buffering agents, colorants, flavorings, solvents, coatings agents, binders, carriers, glidants, lubricants, granulating agents, gelling agents, anti-adherents, preservatives, emulsifiers, antioxidants, plasticizers, surfactants, enteric materials, stabilizing materials, solubilizing materials, adhesive materials, film formers, emollients, dispersing agents, and the like.

An oral pharmaceutical dosage form such as a pill includes any of nano-entity compositions of Formula 1-4 or any combination thereof combined with conventional excipients for tablet, capsule, or other pill-type dosage forms. The pill dosage form may be monolithic or particulate. Typical pill excipients may include binders such as sugars, gelatin, cellulose, starch, methyl cellulose, ethyl cellulose, hydroxypropyl methyl cellulose, and the like. They may also include fillers such as lactose, sucrose, cellulose, calcium carbonate, and the like. The pill may be formulated for extended or immediate release. If needed, the pill may be enteric coated.

An injectable dosage form includes any of the nano-entity compositions of Formula 1-4 in a liquid carrier such as saline, oil, alcohol, or the like, optionally combined with a surfactant to aid solubility or emulsification of the nano-entity.

Some examples of the dosage form have substantially no antibacterial efficacy. Antibacterial efficacy may be measured according to standard USP <51> specifications for antimicrobial effectiveness testing. A compound may be considered as having substantially no antibacterial efficacy, for example, if it exhibits not more than 2 log reduction from the initial bacteria count at 14 days and no increase from the 14 day count to 28 days using USP <51> procedures. By having substantially no antibacterial efficacy, the dosage form may make a safer antifibrotic drug and may be safer for treating fibrotic conditions.

The dosage form may include a therapeutically effective amount of the nano-entity composition, which is the minimum amount that provides the intended therapeutic effect on the patient treated, such as inhibiting collagen synthesis by inhibiting binding of LARP6 with a 5′ stem-loop of a collagen mRNA.

In human patients, examples of therapeutically effective amounts may be 0.1-1,000 mg/day, 0.1-25 mg/day, 1-25 mg/day, 25-50 or less mg/day, 50 or less−75 mg/day, 75-100 mg/day, 100-150 or less mg/day, 150 or less−200 mg/day, 200-250 or less mg/day, 250 or less−300 mg/day, 300−50 or less0 mg/day, 50 or less0-400 mg/day, 400-450 or less mg/day, 450 or less−50 or less0 mg/day, 50 or less0-550 or less mg/day, 550 or less−600 mg/day, 600-650 or less mg/day, 650 or less−700 mg/day, 700-750 or less mg/day, 750 or less−800 mg/day, 800-850 or less mg/day, 850 or less−900 mg/day, 900-950 or less mg/day, 950 or less−1,000 mg/day. Higher doses (1,000-3,000 mg/day) might also be effective.

The therapeutically effective amount by body weight may be 0.001 mg/kg to 20 mg/kg; 5 mg/kg to 15 mg/kg; 1 mg/kg to 5 mg/kg body weight; 0.1 mg/kg to 1 mg/kg; 0.01 mg/kg to 0.1 mg/kg; 0.001 mg/kg to 0.01 mg/kg; or 0.001 to 0.05 mg/kg.

Some particular examples of the pharmaceutical composition may include the following amounts of at least one nano-entity composition of Formulas 1-4: about 0.1 mg, about 1 mg, about 2 mg, about 2.5 mg, about 5 mg, about 10 mg, about 15 mg, about 20 mg, about 25 mg, about 30 mg, about 40 mg, about 45 mg, about 50 or less mg, about 60 mg, about 80 mg, about 100 mg, about 200 mg, or about 250 or less mg.

The values reported above are examples only and are not intended to limit the scope of values.

The pharmaceutical dosage form may be administered once daily or multiple times daily.

The pharmaceutical dosage form may include a combination of different nano-entities of Formulas 1˜4 and may include one or more additional antifibrotic drugs or active ingredients that are therapeutically effective for treating a fibrotic condition.

The pharmaceutical dosage form may be administered as part of a dose regimen that includes varying changes in the dose during the treatment period.

If the pharmaceutical dosage form composition includes a solution of the nano-entities of Formulas 1-4, the nano-entity concentration may be, for example, about 0.01 μM to about 1,000 μM, about 1 μM to about 50 or less0 μM, about 10 μM to about 175 μM, about 10 μM to about 150 or less μM, or about 10 μM to about 125 μM, or about 10 μM to about 100 μM, or about 10 μM to about 25 μM.

The therapeutically effective amount may vary depending on numerous factors, including age, weight, height, severity of the disorder, administration technique, and others. The actual effective amount of the pharmaceutical dosage form administered in a given case to a specific patient may be determined by a medical professional considering the specific patient's situation. The amounts provided above are given as possible examples. In practice, the actual amount of the nano-entities administered to a patient may fall below or above these amounts, depending on the patient's needs.

One or more derivatives of Formulas 1-4 may be used with the proviso that the nano-entity is effective for inhibiting type I collagen production.

The compounds of Formulas 1-4 used in the dosage form may be a particular stereoisomer of Formula 1-4, respectively. If one or more stereo isomers of the respective Formula are determined to be more effective at inhibiting collagen production than other stereo isomers, the % of the more effective stereo isomer used in the dosage form may include at least 80%, at least 90%, or at least 95% of the more active isomer(s).

The pharmaceutical dosage form in in any of its variations discussed above are selected so as to be effective to inhibit binding of LARP6 with the 5′ stem-loop of collagen mRNAs, thereby inhibiting collagen synthesis.

The compound of Formulas 1-4, respectively, used in the dosage form may be a pharmaceutically acceptable salt and/or derivative of the respective Formula so long as the nano-entities including the pharmaceutically acceptable salt and/or derivative is effective for inhibiting type I collagen synthesis.

The nano-entity compositions composed of Formulas 1-4 described above may be used in a method of inhibiting type I collagen synthesis. This method includes contacting at least one cell expressing type I collagen with the nano-entity, where the nano-entity composition is effective for inhibiting type I collagen synthesis by the at least one cell.

“Contacting” may be achieved by placing the nano-entity composition in direct physical association with the cell(s). This is achieved using either a solid, liquid, or gaseous form of a composition containing the nano-entity composition. It includes events that take place both intracellularly and extracellularly and may be accomplished by any of the administration techniques set forth herein or any conventional drug administration technique.

The nano-entity compositions composed of Formulas 1-4 and the pharmaceutical dosage forms described above may be used in a method of treating a patient having a fibrotic condition. The method includes administering to the patient a therapeutically effective amount of a nano-entity composition composed of a plurality of self-associated compounds of Formula 1, Formula 2, Formula 3, and/or Formula 4.

The “patient” may be a human or animal patient that has been identified as having a fibrotic condition.

Examples of fibrotic conditions include but are not limited to a pulmonary fibrosis, a liver fibrosis, a heart fibrosis, a circulatory system fibrosis, a skin fibrosis, a renal fibrosis, and/or an intestinal fibrosis.

There are many different ways the nano-entity composition may be administered to the patient. These administration techniques include oral, sublingual, buccal, intravenous, subcutaneous, transcutaneous, intramuscular, intracutaneous, intrathecal suspensions, tablets, suppositories, capsules, injectables, transdermals, epidural, intraocular, intracranial, inhalation, intranasal, or the like. Any of the pharmaceutical dosage forms described above may be administered to the patient. Any combination of administration techniques may also be used.

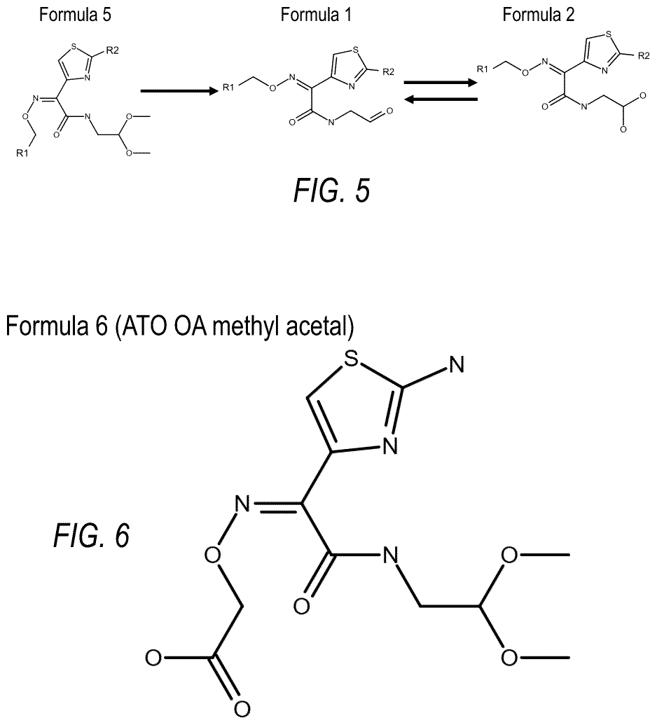

An example of a method of making a nano-entity composition described here is now described referring to FIG. 5. The method includes performing an acid catalyzed hydrolysis of Formula 5 in FIG. 5 to form a tautomeric mixture of Formula 1 and Formula 2.

Formula 5 is a methyl acetal precursor to Formula 1 and Formula 2. R1 and R2 may be as described above with respect to Formula 1 and Formula 2. When Formulas 3 and 4 are being prepared, the specific version of Formula 5 used in the acid hydrolysis reaction is shown in Formula 6 of FIG. 6. The SMILES of Formula 6 is COC(CNC(═O)C(═N/OCC(O)═O)\C1=CSC(N)═N1)OC. Formula 6 may be abbreviated herein as ATO OA methyl acetal.

In the tautomeric mixture, Formula 1 and Formula 2 are as described above with respect to the different variations and identities of R1 and R2. In a specific example of this method, R1 is a carboxylic acid and R2 is an amine. In another specific example of this method Formula 1 is Formula 3 and Formula 2 is Formula 4.

Some specific examples of the acid catalyzed hydrolysis step are performed at a pH of 1-4 and a temperature of 45 C-90 C. A more specific example of the acid hydrolysis step is performed at a pH of 3 and a temperature of 60 C.

The tautomeric mixture is dissolved in a solvent to form a nano-entity composed of a plurality of self-associated compounds of the Formula 1 and Formula 2. The solvent may vary in this step. A particular solvent useful in this step is dimethyl sulfoxide (DMSO).

The nano-entity composed of the plurality of self-associated compounds of Formula 1 and Formula 2 are subsequently precipitated from the tautomeric mixture solution. This precipitation step is accomplished using a solvent capable of precipitating the nano-entity. In this step a precipitating agent that renders the nano-entity insoluble in the solvent is used. An example of a precipitating agent is an alcohol, such as methanol, which is added in excess with respect to the solvent. In a particular example of this method the solvent is DMSO and the precipitating agent is methanol in 19 fold volume excess with respect to the DMSO.

The precipitated nano-entity is collected via filtration or the like and is then washed.

The precipitated nano-entity is then dissolved in an aqueous buffer solution having a pH of at least 6, 6-14, 6-13, 6-12, 6-11, 6-10, or 6-9.

### Examples

This section provides an example of the nano-entity composition for illustration purposes and is not intended to limit the scope of what may be claimed.

Multi-unit nano-entities are formed by self-association of the compound during its conversion from methyl-acetal into aldehyde. ATO-OA (Formula 3) is in equilibrium with ATO-OA hydride (Formula 4) in aqueous solutions. The self-association of Formula 3 and Formula 4 are driven by formation of H-bonds between numerous H-bond donors and acceptors, present on each molecule. Formula 3 has eight H-bond donors and acceptors. Formula 4 has nine H-bond donors and acceptors. H-bonds can be intermolecular and intramolecular, the former are responsible for formation of NE.

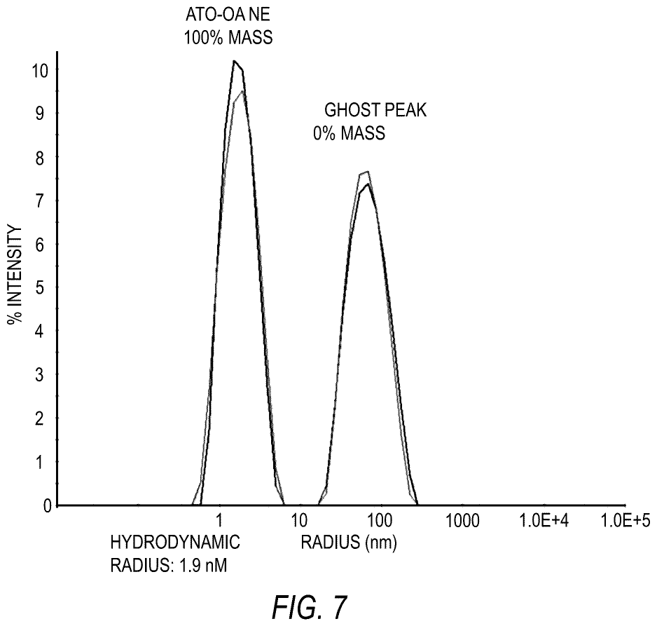

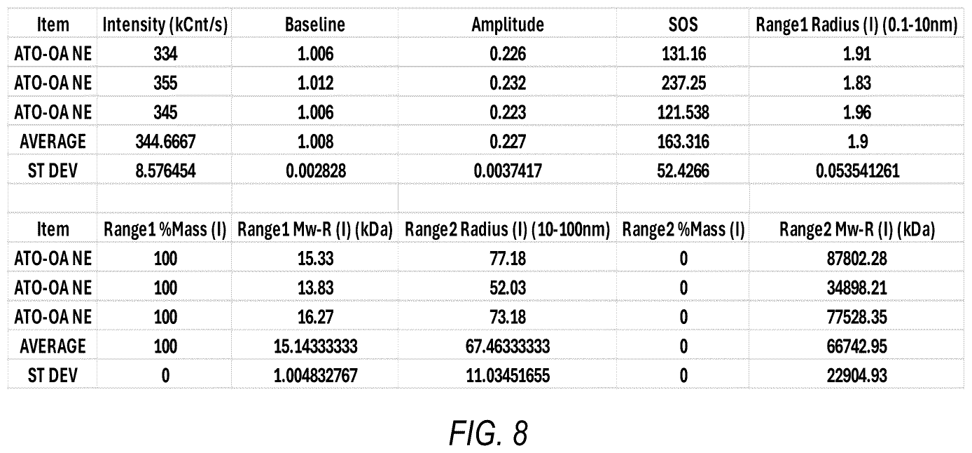

FIG. 7 shows the result of dynamic light scattering (DLS) measurements of the hydrodynamic radius of nano-entities composed of Formula 3 and Formula 4, which are referred to in the Examples as ATO-OA NE. The mean hydrodynamic radius was 1.9 nM, which corresponds to a molecular mass of ˜15 kD, assuming a globular shape with occupancy in the globular interior. Assembly of ˜50 individual molecules is needed to form a fully filled globular ATO-OA NE of 15 kD. Therefore, ˜50 molecules were considered to be the maximum number of molecules in an average ATO-OA NE in the measurement. A smaller number of molecules can be inferred if the interior of the ATO-OA NE globe is hollow. A ghost peak associated with no mass was also detected, indicating the presence of dust or other very large particles in the sample. The parameters of DLS measurements are shown as a table in FIG. 8.

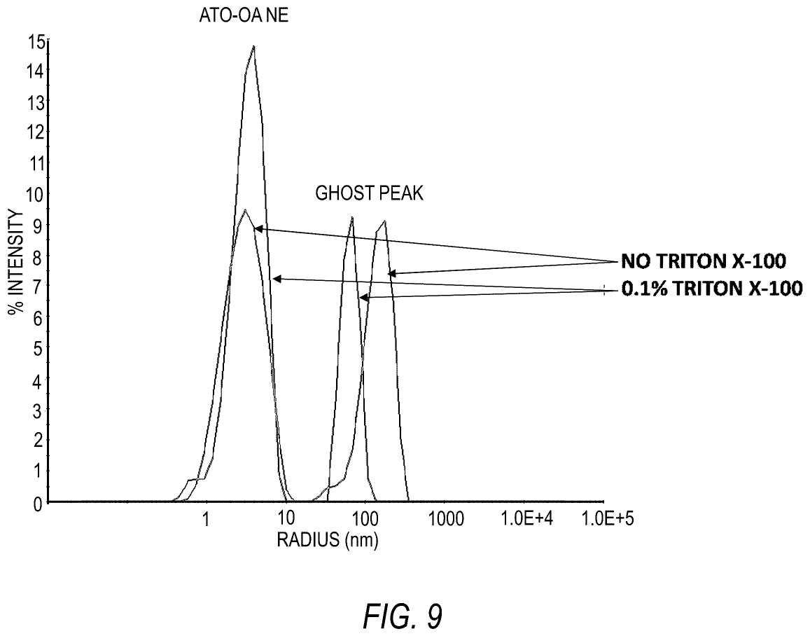

FIG. 9 is data showing the comparison of the hydrodynamic radius of ATO-OA NE without detergent and in the presence of 0.1% Triton X-100. There was no change of the hydrodynamic radius of the ATO-OA NE, suggesting ATO-OA NE is not composed of colloidal particles.

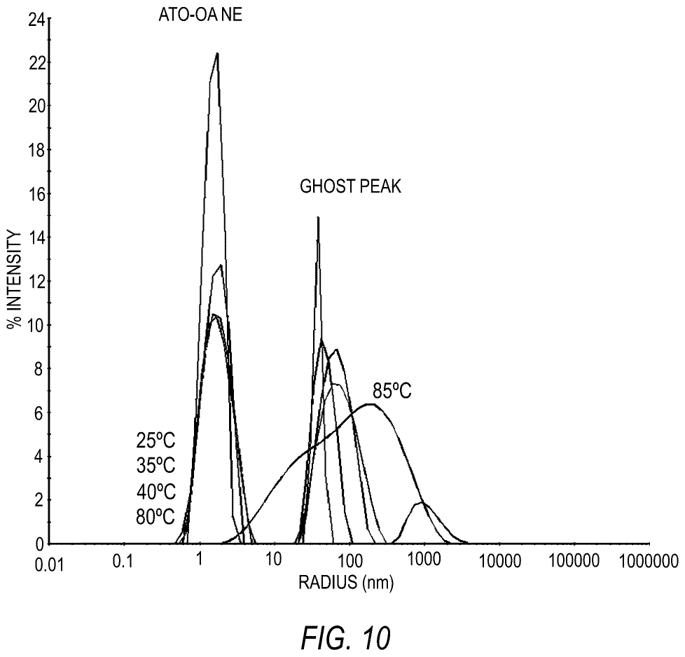

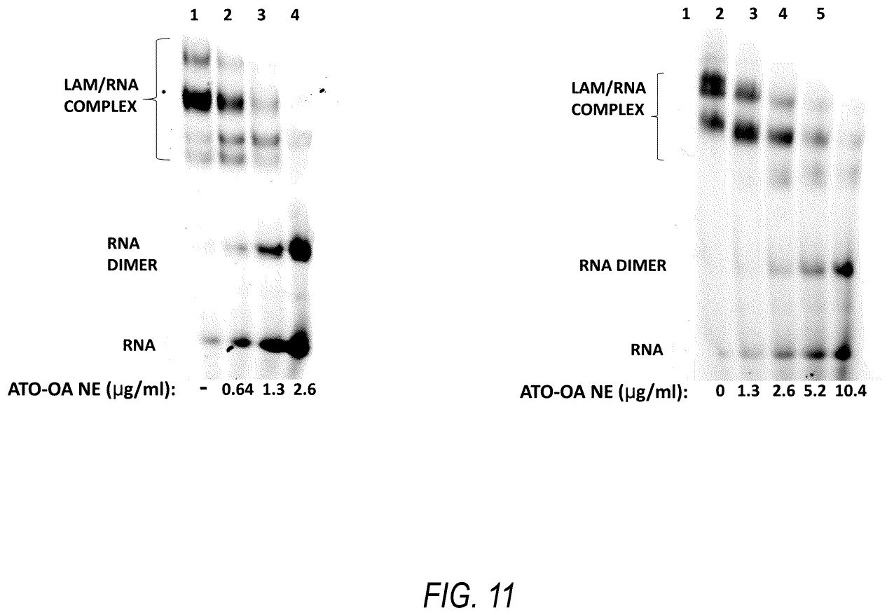

FIG. 10 is data showing ATO-OA NE is stable at elevated temperatures. There was no change in the hydrodynamic radius of ATO-OA NE with heating up to 80 C. At 85 C, the hydrodynamic radius of ATO-OA NE broadened.

The size of the ATO-OA NE can be polymorphic, but their average hydrodynamic radius corresponds to the molecular weight of 15 kD, assuming a globular shape with the occupied interior. This indicates the average ATO-OA NE are formed by assembly of ˜50 of individual molecules or possibly less if the interior is unoccupied.

ATO-OA NE dissociated LARP6 from its target RNA sequence in type I procollagen mRNAs. Type I procollagen is composed of three polypeptides, two COL1A1 and one COL1A2, which are encoded by two mRNAs, COL1A1 and COL1A2. Both type I procollagen mRNAs have a very similar, 46-48-nucleotide long, sequence in their 5′UTR, termed the 5′ stem-loop (5′SL). The 5′SL binds LARP6 protein with high affinity and sequence specificity and COL1A1 5′SL and COL1A2 5′SL have similar affinities for LARP6. To assess if ATO-OA NE can disrupt the LARP6/5′SL RNA binding in vitro, a truncated version of LARP6 (amino-acids 73-303, LAM) was used which contains two domains of LARP6, the LA domain and the RRM domain. LAM is necessary and sufficient for binding the 5′SL with high affinity, however, LA domain alone can bind 5′SL with lower affinity.

FIG. 11, left panel, shows the binding of LAM to A1 5′SL RNA in the presence of increasing concentrations of ATO-OA NE. The concentration of ATO-OA NE was given as μg/ml, because of the variable size of ATO-OA NE. The electrophoretic mobility shift assay (EMSA) used here resolved the LAM/5′SL RNA complexes from the free 5′SL RNA. With 0.64 μg/ml of ATO-OA NE the LAM binding was reduced (lane 2), it was further diminished by 1.3 μg/ml (lane 3) and almost completely abolished by 2.6 μg/ml (lane 4). The increase in free RNA monomer and dimer paralleled the dissociation of LAM/RNA complex.

To exclude the chance the inhibitory activity of ATO-OA NE may be due to a colloidal nature of ATO OA NE, the dose response experiment was repeated in the presence of detergent (FIG. 11, right panel). Triton-X100 at 0.1% dissolved colloidal other nano-entities, but had only minimal effect on ATO-OA NE activity (lanes 4-6). This confirmed that the ATO-OA NE are not colloidal particles.

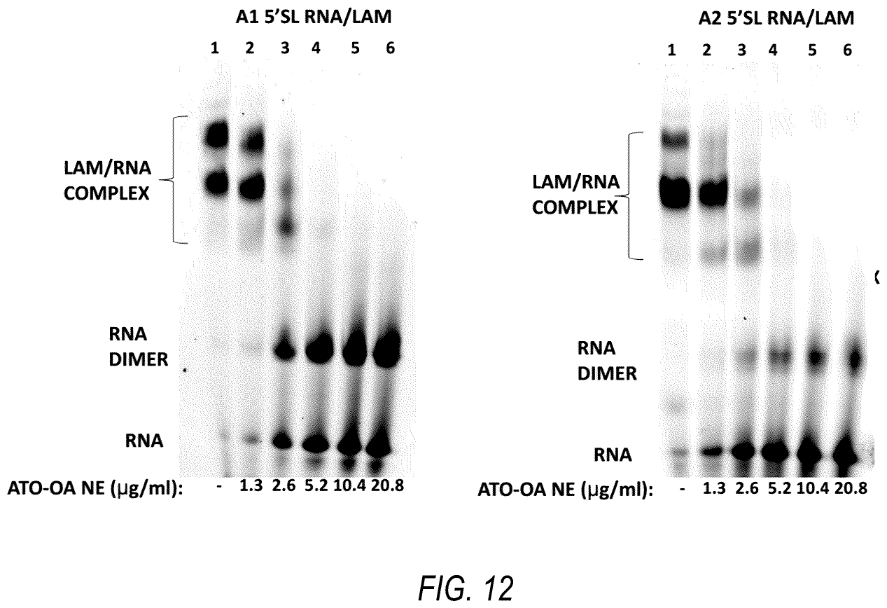

FIG. 12 compares the inhibitory activity of ATO-OA NE for LAM binding to COL1A1 5′SL (left panel) and to COL1A2 5′SL (right panel). ATO-OA NE inhibited LAM binding to both 5′SL RNA targets at the same concentrations. These experiments indicated that ATO-OA NE is equally effective in abrogating LARP6 interaction with 5′SL of COL1A1 and COL1A2 mRNAs.

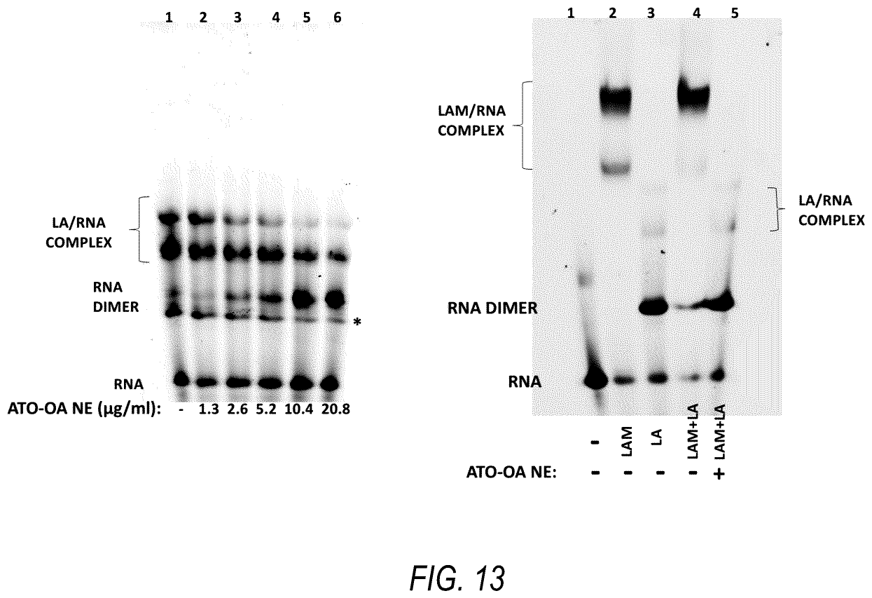

FIG. 13 shows that ATO-OA NE does not inhibit binding of the LA domain alone. The LA domain alone can bind 5′SL RNA with lower affinity than LAM. LAM contains LA and RRM, which increases the binding affinity. Because the LA domain alone can bind 5′SL RNA, ATO-OA NE activity towards binding of this domain could be assessed.

Increasing concentrations of ATO-OA NE to the binding reactions containing LA showed minimal inhibition at concentrations of 10.4 μg/ml or higher (FIG. 13, left panel, lanes 5 and 6), which are >10-fold higher concentrations than for inhibition of LAM binding.

When both, LAM and LA, were present in the same binding reaction at equimolar concentrations, ATO-OA NE abrogated only the LAM/5′SL complex without affecting the LA/5′SL complex. FIG. 13, right panel, shows binding of LAM alone (lane 2), binding of LA alone (lane 3) and binding when both proteins were present, where binding of LAM predominated (lane 4). When ATO-OA NE was added to this reaction, the LAM binding was abrogated and the LA binding reappeared (lane 5), suggesting the specific inhibition of high affinity LAM binding. Because LAM also contains RRM domain, the presence of RRM domain is critical for inhibitory activity of ATO-OA NE.

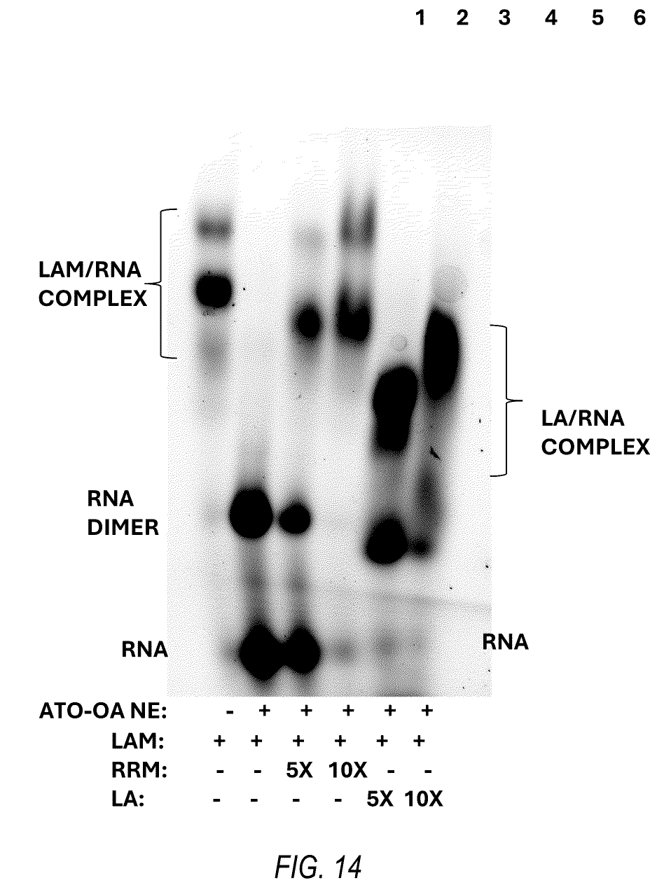

FIG. 14 shows that ATO-OA NE was sequestered by RRM. In FIG. 14, LAM was first bound to A1 5′SL RNA (lane 1), then ATO-OA NE was added to inhibit binding of LAM (lane 2). When 5 and 10 molar excess of RRM was added, the inhibition of LAM binding was relieved and LAM/A1RNA complex was restored (lanes 3 and 4). This suggested that the excess of RRM sequestered ATO-OA NE.

The excess of LA domain, which is not responsive to ATO-OA NE, could not relieve the inhibition of LAM/5′SL complex. Instead, the LA domain formed its own complexes with 5′SL RNA (lanes 5 and 6), which were not affected by ATO-OA NE. This suggested that ATO-OA NE targets the RRM domain of LARP6 and that this compromises LARP6 ability to bind 5′SL RNA.

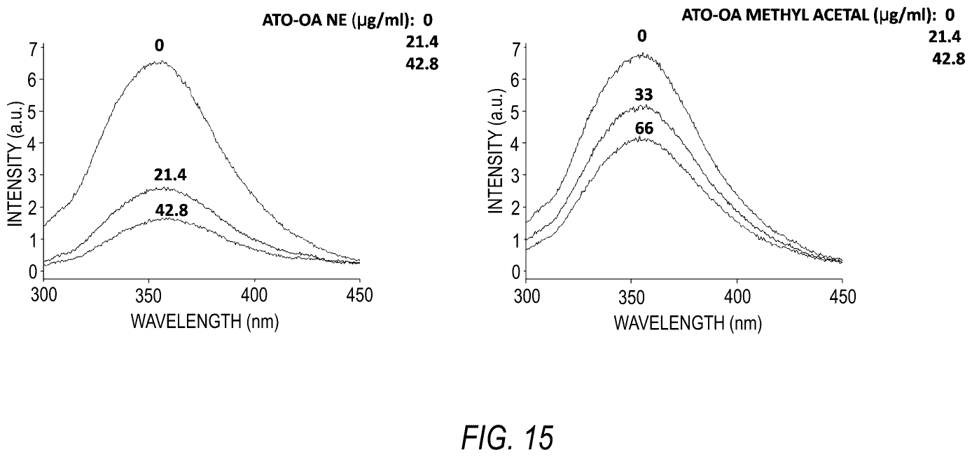

FIG. 15 is data showing that ATO-OA NE changes the conformation of the RRM domain of LARP6. RRM domain was incubated with increasing concentrations of ATO-OA NE (left panel) or ATO-OA methyl acetal (Formula 5) (right panel) and the intensity of intrinsic tryptophan fluorescence of RRM was measured. The strong quenching of fluorescence by ATO-OA NE suggests conformational alteration of the RRM domain, while ATO-OA methyl acetal is inactive and showed only a nonspecific inner filter effect.

The formation of ATO-OA NE from Formula 3 and Formula 4 was needed for therapeutic effectiveness. ATO-OA NE specifically interacts with the RRM domain of LARP6 to change its conformation. The altered RRM conformation obliterates the ability of LARP6 to recognize 5′SL of type I collagen mRNAs. ATO-OA NE can also dissociate the already bound LARP6. Inhibition of LARP6 results in impaired assembly and secretion of type I procollagen and reduces its deposition in the extracellular space. This is the basis of antifibrotic activity of ATO-OA NE.

The chemical structures provided herein are intended to generally illustrate the structure of the molecule and are not intended to be limited thereto. Instead, the structures are provided to show where the general constituents are located on each molecule. For example, the structures are not intended to be limited to the particular compound shown.

This disclosure describes certain examples and features, but not all possible examples and features of the compositions and methods. Where a particular feature is disclosed in the context of a particular example, that feature can also be used, to the extent possible, in combination with and/or in the context of other examples. The methods and compositions may be embodied in many different forms and should not be construed as limited to only the examples and features described here.

1. A composition comprising a pharmaceutical dosage form including a nano-entity composed of a plurality of self-associated compounds of a first formula 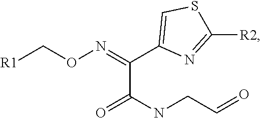
wherein:
R1 and R2 are independently selected from the group consisting of H, an alkyl group, an alkenyl group, an alkynyl group, an aryl group, an alkoxy group, a hydroxy group, a carboxy group, an acyl group, a nitro group, a phosphor group, a halo group, a sulfo group, an ester group, an ether group, an amine group, and a combination thereof;
wherein the pharmaceutical dosage form also includes a tautomer of the first formula.

2. The composition of claim 1, wherein R1 is a carboxyl group.

3. The composition of claim 1, wherein R2 is an amine group.

4. (canceled)

5. The composition of claim 1, wherein the nano-entity is also composed of a plurality of self-associated compounds of a second formula

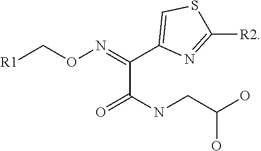

6. The composition of claim 1, wherein the first formula is

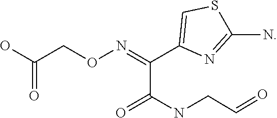

7. (canceled)

8. The composition of claim 1, wherein the pharmaceutical dosage form is effective to inhibit collagen synthesis by inhibiting binding of LARP6 with a 5′ stem-loop of a collagen mRNA.

9. The composition of claim 1, wherein the pharmaceutical dosage form is at least one of an oral dosage form and an injectable dosage form.

10. A method comprising:
administering to a patient having a fibrotic condition a therapeutically effective amount of the composition of claim 1 to the patient.

11. The method of claim 10, wherein the fibrotic condition is at least one of a pulmonary fibrosis, a liver fibrosis, a heart fibrosis, a circulatory system fibrosis, a skin fibrosis, a renal fibrosis, and/or an intestinal fibrosis.

12. The method of claim 10, wherein administering is oral administration and/or by injection.

13. The method of claim 10, wherein R1 is a carboxyl group.

14. The method of claim 10, wherein R2 is an amine group.

15. The method of claim 10, wherein the pharmaceutical dosage form also includes a tautomer of the first formula.

16. The method of claim 10, wherein the nano-entity is also composed of a plurality of self-associated compounds of a second formula

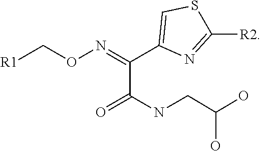

17. The method of claim 10, wherein the first formula is

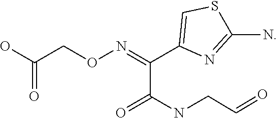

18. (canceled)

19. A method comprising:
performing an acid catalyzed hydrolysis of a first formula 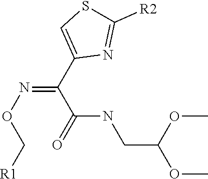
to form a tautomeric mixture of a second formula 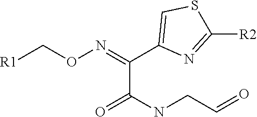
and a third formula 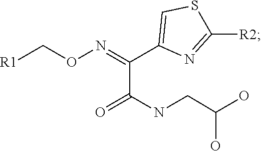
and
dissolving the tautomeric mixture in a solvent to form a nano-entity composed of a plurality of self-associated compounds of the second formula and the third formula;
wherein R1 and R2 are independently selected from the group consisting of H, an alkyl group, an alkenyl group, an alkynyl group, an aryl group, an alkoxy group, a hydroxy group, a carboxy group, an acyl group, a nitro group, a phosphor group, a halo group, a sulfo group, an ester group, an ether group, an amine group, and a combination thereof.

20. The method of claim 19, further comprising:
precipitating the nano-entity; and
dissolving the precipitated nano-entity in an aqueous solution having a pH of 6-14.

21. The method of claim 19, wherein the solvent is dimethyl sulfoxide.

22. The method of claim 19, wherein the acid catalyzed hydrolysis is performed at a pH of 1-4 and a temperature of 45 C to 90 C.
